(19) United States
(12) Patent Application Publication
(10) Pub. No.: US 2023/0000822 A1
(43) Pub. Date: Jan. 5, 2023

(54) A COMBINATION OF A BTK INHIBITOR AND ABATACEPT FOR THE TREATMENT OF RHEUMATOID ARTHRITIS

(71) Applicant: BRISTOL-MYERS SQUIBB COMPANY, Princeton, NJ (US)

(72) Inventor: Dennis Michael Grasela, Huntingdon Valley, PA (US)

(21) Appl. No.: 17/779,328

(22) PCT Filed: Nov. 24, 2020
(86) PCT No.: PCT/US2020/061905

§ 371 Date: May 24, 2022

Related U.S. Application Data
(60) Provisional application No. 62/939,776, filed on Nov. 25, 2019.

(51) Int. Cl. A61K 31/404; A61K 9/00; C07K 16/28

(52) U.S. Cl. CPC A61K 31/404; A61K 9/0019; C07K 16/28

(57) ABSTRACT

Disclosed is method of treating a patient having rheumatoid arthritis, comprising administering sequentially to said patient, a therapeutically effective dose of branebrutinib for a first period, followed by a therapeutically effect dose of abatacept for a second period.

### CROSS REFERENCE

This application claims the benefit of U.S. Provisional Application Ser. No. 62/939,776 filed Nov. 25, 2019 which is incorporated herein in its entirety.

### DESCRIPTION

The present invention generally relates methods of treating a patient having rheumatoid arthritis, comprising administering sequentially to said patient, a therapeutically effective dose of a Btk inhibitor for a first period, followed by a therapeutically effect dose of abatacept for a second period.

### BACKGROUND OF THE INVENTION

Rheumatoid arthritis (RA) is a chronic disease affecting 0.5 to 1.0% of the population in which diarthrodial (synovial) joints become inflamed, usually with a bilateral symmetric pattern, and with a high incidence of damage and disability. RA is a systematic disease, and manifestations may include constitutional symptoms such as fatigue, fever and malaise, vasculitis, cardiovascular complications, ocular involvement, pulmonary interstitial disease, and widespread nodulosis. Patients with RA have a reduced life span which correlates with disease severity, and increased levels of cytokines such as TNF-α, IL-1 and IL-6, circulating immune complexes (ICs), rheumatoid factor (RF) levels, and anti-citrullinated protein antibodies (ACPAs).

Despite recent progress in RA therapy with a number of approved targeted biologics in the United States and Europe, unmet medical needs remain. First, many agent approved for RA exhibit significant safety concerns, including risk of tuberculosis and other serious infections, malignancies, and gastrointestinal perforation. Second, many patients are only partially respond to current available therapies, and true remission is achieved by only a minority of patients (<10% in many series). Third, current treatments have toxicities that lead to frequent drug discontinuation. In addition, the destructive process of RA cannot be halted in all patients, and new drugs that reduce osteoclast-mediated bone loss and inflammation may offer unique benefits to patients with RA.

Immune complexes containing IgG play a critical role in the immunopathology of many immune-mediated disorders. In RA, immune complexes (ICs) are present in the joints and act on synovial macrophages to drive the production of the cytokines, chemokines, and matrix metalloproteinases (MMPs) that are critical in mediating disease pathology. Expression of FcγRIIa and FcγRIIIa are increased in monocytes and macrophages of patients with RA, and produce higher levels of TNF-α and MMPs than healthy controls.

Activating Fcγ receptors are also important in the activation of monocyte-derived dendritic cells. Genome-wide association studies have shown FcγRIIIa to be associated with RA susceptibility, and murine models of RA such as the collagen-induced arthritis (CIA) model have demonstrated a role for activation Fcγ receptors in disease pathogenesis. BTK is highly expressed in myeloid lineages and regulates signaling pathways leading from the binding of ICs to FcγRIIIa and FcγRIIa to expression of pro-inflammatory cytokines, chemokines, and cell adhesion molecules. BTK also mediated FcγRI signaling in mast cells and basophils although the role of these pathways in RA is not well established. However, IgE ACPAs have been identified in patients with RA, and the number of activated mast cells may be increased in synovial tissue and have been correlated with disease activity. Thus, inhibition of BTK could have therapeutic benefit on a variety of immunopathogenic features of RA.

Bruton's tyrosine kinase (BTK) is a member of the Tec family of non-receptor tyrosine kinases and is expressed in all hematopoietic cells, with the exception of T cells and terminally differentiated plasma cells. Wu, J. et al., Journal of Hematology & Oncology (2016) 9:80.

Lastly, BTK inhibition could potentially counter the bone damage in RA through its role in mediating RANK-dependent osteoclastogenesis.

The inventors have discovered methods of treating a patient having rheumatoid arthritis by the administering sequentially a BTK inhibitor for a first period followed by abatacept for a second period.

### SUMMARY OF THE INVENTION

The present invention provides methods of treating a patient having rheumatoid arthritis, comprising administering sequentially to said patient, a therapeutically effective dose of a Btk inhibitor for a first period, followed by a therapeutically effect dose of abatacept for a second period.

The present invention provides methods of treating a patient having rheumatoid arthritis, comprising administering sequentially to said patient, a therapeutically effective dose of an irreversible Btk inhibitor for a first period, followed by a therapeutically effect dose of abatacept for a second period.

The present invention provides methods of treating a patient having rheumatoid arthritis, comprising administering sequentially to said patient, a therapeutically effective dose of branebrutinib for a first period, followed by a therapeutically effect dose of abatacept for a second period.

These and other features of the invention will be set forth in expanded form as the disclosure continues.

### DEFINITIONS

In order that the present description may be more readily understood, certain terms are first defined. Additional definitions are set forth throughout the detailed description.

A “BTK inhibitor” inhibits the function of BTK. BTK inhibitors can associate with BTK reversibly or irreversibly, and include antibodies, small molecules, and millimolecular compounds.

An “irreversible BTK inhibitor” can be a small molecule inhibitor of BTK that forms a covalent chemical bond with BTK. Irreversible BTK inhibitors, such as branebrutinib, ibrutinib, acalabrutinib, evobrutinib, spebrutinib, and zanubrutinib, target the positioned noncatalytic cysteine residue (Cys481) in the kinase domain.

“Branebrutinib” is an oral, highly selective, irreversible inhibitor of BTK, having the structure:

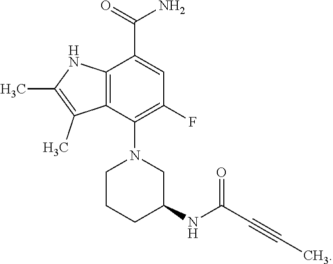 [SMILES: CC#CC(=O)N[C@H]1CCCN(c2c(F)cc(C(N)=O)c3[nH]c(C)c(C)c23)C1]

The chemical name for branebrutinib is (S)-4-(3-(but-2-ynamido)piperidin-1-yl)-5-fluoro-2,3-dimethyl-1H-indole-7-carboxamide. The discovery and synthesis of branebrutinib is described in Watterson, S. H., et al. J. Med. Chem. 2019, 62, 3228-3250.

“Abatacept” is a biological compound approved for the treatment of patients with active RA. Abatacept is a selective T cell costimulation modulator. The biological compound is a soluble fusion protein that consists of the extracellular domain of human cytotoxic T-lymphocyte associated antigen 4 (CTLA-4) linked to the modified Fc (hinge, CH2, and CH3 domains) portion of human immunoglobulin G1 (IgG1). Abatacept is produced by recombinant DNA technology in a mammalian cell expression system. The apparent molecular weight of abatacept is 92 kilo Daltons. Abatacept is marketed as ORENCIA®.

As used herein, “administering” refers to the physical introduction of a composition comprising a therapeutic agent to a patient, using any of the various methods and delivery systems known to those skilled in the art.

Suitable routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intra-arterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “treat,” “treating,” and “treatment,” as used herein, refer to any type of intervention or process performed on, or administering an active agent to, the patient with the objective of reversing, alleviating, ameliorating, inhibiting, or slowing down or preventing the progression, development, severity or recurrence of a symptom, complication, condition or biochemical indicia associated with a disease.

The term “therapeutically effective amount” or “therapeutically effective dosage” of a drug or therapeutic agent is any amount of the drug that, when used alone promotes disease regression evidenced by a decrease in severity of disease symptoms, an increase in frequency and duration of disease symptom-free periods, or a prevention of impairment or disability due to the disease affliction. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

The term “patient” includes human and other mammalian subjects that receive therapeutic treatment.

Branebrutinib can be administered orally, mucosally, or parentally including intravascularly, intravenously, intraperitoneally, subcutaneously, intramuscularly, and intrasternally in dosage unit formulations containing conventional pharmaceutically acceptable carriers, adjuvants, and vehicles. For oral administration, the pharmaceutical composition may be in the form of, for example, a tablet, capsules such as hard gelatin capsules or soft gelatin capsules, liquid capsule, suspensions including aqueous suspensions or oily suspensions, liquids, and emulsions. Pharmaceutical compositions intended for oral administration can be prepared according to any methods known in the art for manufacturing pharmaceutical compositions intended for oral administration.

### DETAILED DESCRIPTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Provided herein are methods of treating a patient having rheumatoid arthritis. The methods comprise administering sequentially to a patient:

(i) a therapeutically effective dose of branebrutinib for a first period; and

(ii) a therapeutically effect dose of abatacept for a second period.

The first period and second period are sequential and do not overlap. The second period starts after the completion of the first period.

In one embodiment the first period is from 1 to 200 days. Included in this embodiment is a first period selected from 1 to 175 days; 1 to 140 days; 1 to 126 days; 1 to 112 days; 1 to 98 days; 1 to 84 days; 1 to 70 days; 1 to 56 days; 1 to 49 days; 1 to 42 days; 1 to 35 days; 1 to 28 days; and 1 to 14 days.

In one embodiment the first period is from 7 to 200 days. Included in this embodiment is a first period selected from 7 to 175 days; 7 to 140 days; 7 to 126 days; 7 to 112 days; 7 to 98 days; 7 to 84 days; 7 to 70 days; 7 to 56 days; 7 to 49 days; 7 to 42 days; 7 to 35 days; 7 to 28 days; and 7 to 14 days.

In one embodiment the first period is from 14 to 200 days. Included in this embodiment is a first period selected from 14 to 175 days; 14 to 140 days; 14 to 126 days; 14 to 112 days; 14 to 98 days; 14 to 84 days; 14 to 70 days; 14 to 56 days; 14 to 49 days;

14 to 42 days; 14 to 35 days; and 14 to 28 days.

In one embodiment the first period is from 21 to 200 days. Included in this embodiment is a first period selected from 21 to 175 days; 21 to 140 days; 21 to 126 days; 21 to 112 days; 21 to 98 days; 21 to 84 days; 21 to 70 days; 21 to 56 days; 21 to 49 days; 21 to 42 days; 21 to 35 days; and 21 to 28 days.

In one embodiment the second period is at least 2 weeks. Included in this embodiment is a second period selected from at least 4 weeks; at least 6 weeks; at least 8 weeks; at least 10 weeks; at least 12 weeks; at least 14 weeks; at least 16 weeks; at least 18 weeks; and at least 20 weeks. The second period can be continued for any period of time provided that the patient responds to treatment. Response to treatment, which can be determined by a qualified medical professional, includes reversing, alleviating, ameliorating, inhibiting, or slowing down or preventing the progression, development, severity or recurrence of a symptom, complication, condition or biochemical indicia associated with rheumatoid arthritis. The second period can extend as long as the remaining lifetime of the patient.

In one embodiment, the abatacept therapy in the second period is continued for the period of time that the patient responds to treatment as determined by a qualified medical professional.

In one embodiment the second period is from at least 2 weeks to 20 years. Included in this embodiment is a second period selected from at least 2 weeks to 15 years; from at least 2 weeks to 10 years; from at least 2 weeks to 5 years; from at least 2 weeks to 3 years; from at least 2 weeks to 2 years; from at least 2 weeks to 1 year; from at least 2 weeks to 40 weeks; from at least 2 weeks to 30 weeks; from at least 2 weeks to 20 weeks; from at least 2 weeks to 12 weeks; and from at least 2 weeks to 10 weeks.

In one embodiment the second period is at least 4 weeks. Included in this embodiment is a second period selected from at least 4 weeks to 20 years; from at least 4 weeks to 15 years; from at least 4 weeks to 10 years; from at least 4 weeks to 5 years;

least 4 weeks to 4 years; from at least 4 weeks to 3 years; from at least 4 weeks to 2 years; from at least 4 weeks to 1 year; from at least 4 weeks to 40 weeks; from at least 4 weeks to 30 weeks; from at least 4 weeks to 20 weeks; from at least 4 weeks to 12 weeks; and from at least 4 weeks to 10 weeks.

In one embodiment the second period is at least 8 weeks. Included in this embodiment is a second period selected from at least 8 weeks to 20 years; from at least 8 weeks to 15 years; from at least 8 weeks to 10 years; from at least 8 weeks to 5 years; from at least 8 weeks to 4 years; from at least 8 weeks to 3 years; from at least 8 weeks to 2 years; from at least 8 weeks; from at least 8 weeks to 1 year; from at least 8 weeks to 40 weeks; from at least 8 weeks to 30 weeks; from at least 8 weeks to 20 weeks; from at least 8 weeks to 12 weeks; and from at least 8 weeks to 10 weeks.

In one embodiment the second period is at least 12 weeks. Included in this embodiment is a second period selected from at least 12 weeks to 20 years; from at least 12 weeks to 15 years; from at least 12 weeks to 10 years; from at least 12 weeks to 5 years; from at least 12 weeks to 4 years; from at least 12 weeks to 3 years; from at least 12 weeks to 2 years; from at least 12 weeks; from at least 12 weeks to 1 year; from at least 12 weeks to 40 weeks; from at least 12 weeks to 30 weeks; and from at least 12 weeks to 20 weeks.

In one embodiment the second period is at least 16 weeks. Included in this embodiment is a second period selected from at least 16 weeks to 20 years; from at least 16 weeks to 15 years; from at least 16 weeks to 10 years; from at least 16 weeks to 5 years; from at least 16 weeks to 4 years; from at least 16 weeks to 3 years; from at least 16 weeks to 2 years; from at least 16 weeks; from at least 16 weeks to 1 year; from at least 16 weeks to 40 weeks; from at least 16 weeks to 30 weeks; and from at least 16 weeks to 20 weeks.

In one embodiment the second period is at least 20 weeks. Included in this embodiment is a second period selected from at least 20 weeks to 20 years; from at least 20 weeks to 15 years; from at least 20 weeks to 10 years; from at least 20 weeks to 5 years; from at least 20 weeks to 4 years; from at least 20 weeks to 3 years; from at least 20 weeks to 2 years; from at least 20 weeks; from at least 20 weeks to 1 year; from at least 20 weeks to 40 weeks; and from at least 20 weeks to 30 weeks.

In one embodiment, a therapeutically effective dose of branebrutinib is in the range of 0.5 to 10 mg/day. Included in this embodiment is a therapeutically effective dose of branebrutinib selected from the ranges of 1 to 10 mg/day; 2 to 10 mg/day; 3 to 10 mg/day; 4 to 10 mg/day; 5 to 10 mg/day; 6 to 10 mg/day; 7 to 10 mg/day; 8 to 10 mg/day; and 9 to 10 mg/day.

In one embodiment, a therapeutically effective dose of branebrutinib is 0.5 mg/day.

In one embodiment, a therapeutically effective dose of branebrutinib is 1 mg/day.

In one embodiment, a therapeutically effective dose of branebrutinib is 1.5 mg/day.

In one embodiment, a therapeutically effective dose of branebrutinib is 2 mg/day.

In one embodiment, a therapeutically effective dose of branebrutinib is 2.5 mg/day.

In one embodiment, a therapeutically effective dose of branebrutinib is 3 mg/day.

In one embodiment, a therapeutically effective dose of branebrutinib is 3.5 mg/day.

In one embodiment, a therapeutically effective dose of branebrutinib is 4 mg/day.

In one embodiment, a therapeutically effective dose of branebrutinib is 4.5 mg/day.

In one embodiment, a therapeutically effective dose of branebrutinib is 5 mg/day.

In one embodiment, a therapeutically effective dose of branebrutinib is 5.5 mg/day.

In one embodiment, a therapeutically effective dose of branebrutinib is 6 mg/day.

In one embodiment, a therapeutically effective dose of branebrutinib is 6.5 mg/day.

In one embodiment, a therapeutically effective dose of branebrutinib is 7 mg/day.

In one embodiment, a therapeutically effective dose of branebrutinib is 7.5 mg/day.

In one embodiment, a therapeutically effective dose of branebrutinib is 8 mg/day.

In one embodiment, a therapeutically effective dose of branebrutinib is 8.5 mg/day.

In one embodiment, a therapeutically effective dose of branebrutinib is 9 mg/day.

In one embodiment, a therapeutically effective dose of branebrutinib is 9.5 mg/day.

In one embodiment, a therapeutically effective dose of branebrutinib is 10 mg/day.

The therapeutically effective dose of branebrutinib can be administered as a single daily dose (q.d.), divided and administered twice daily (b.i.d.), or divided and administered as three or more doses per day.

In one embodiment, a therapeutically effective dose of branebrutinib is administered as a single daily dose.

In one embodiment, a therapeutically effective dose of branebrutinib is administered as a twice daily dose. For example, a therapeutically effective dose of 6 mg/day of branebrutinib can be administered as 3 mg dose administered twice daily (b.i.d).

Abatacept is available as:

### Intravenous Infusion

For Injection: 250 mg lyophilized powder in a single-use vial for reconstitution and dilution prior to intravenous infusion.

### Subcutaneous Injection

Injection: 50 mg/0.4 mL, 87.5 mg/0.7 mL, 125 mg/mL solution in single-dose prefilled syringes for subcutaneous use.
Injection: 125 mg/mL solution in a single-dose prefilled ClickJect™ autoinjector for subcutaneous use.

Abatacept Dosage and Administration

Intravenous Administration for Adult RA and Adult PsA

| Body Weight of Patient | Dose | Number of Vials |
|---|---|---|
| Less than 60 kg | 500 mg | 2 |
| 60 to 100 kg | 750 mg | 3 |
| More than 100 kg | 1000 mg | 4 |

### Subcutaneous Administration for Adult RA

Administer by subcutaneous injection once weekly with or without an intravenous loading dose. For patients initiating therapy with an intravenous loading dose, administer a single intravenous infusion (as per body weight categories above), followed by the first 125 mg subcutaneous injection given within a day of the intravenous infusion.
Patients transitioning from ORENCIA intravenous therapy to subcutaneous administration should administer the first subcutaneous dose instead of the next scheduled intravenous dose.

In one embodiment, the patient is a human.

In one embodiment, the abatacept therapy administered during the second period is continued for the period of time that the patient responds to treatment as determined by a qualified medical professional. Response to treatment includes reversing, alleviating, ameliorating, inhibiting, or slowing down or preventing the progression, development, severity or recurrence of a symptom, complication, condition or biochemical indicia associated with rheumatoid arthritis. The second period can extend as long as the remaining lifetime of the patient.

### RA Protocol Duration:

The total duration of participation in the RA protocol is approximately 32 weeks and will be divided into the following periods: screening (up to 4 weeks), double-blind, PBO-controlled branebrutinib treatment for 12 weeks (Week 0 to Week 12), open-label treatment with abatacept for an additional 12 weeks (Week 12 to Week 24), and follow-up (4 weeks).

### RA Sub-Protocol Study Treatment:

Branebrutinib followed by open-label abatacept therapy: Day 1 to Week 24
Medication Potency IP/Non-IP
Branebrutinib: dose ranges above QD, PO IP
Branebrutinib PBO: QD, PO IP
Abatacept: 125 mg QW, SC IP
IP=investigational product; PBO=placebo; PO=administered orally; QD=once daily; QW=once weekly; SC=subcutaneous
ACR50 response at Week 24 compared to baseline

To evaluate the efficacy at Week 24 of branebrutinib or PBO treatment followed by abatacept treatment in subjects with moderate to severe RA on a stable background of methotrexate (MTX) who have had an inadequate response to MTX.

The ACR50 is a composite measure defined as both improvement of 20% in the number of tender and number of swollen joints, and a 50% improvement in three of the following five criteria: patient global assessment, physician global assessment, functional ability measure [most often Health Assessment Questionnaire (HAQ)], visual analog pain scale, and erythrocyte sedimentation rate or C-reactive protein (CRP).

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

What is claimed is:

1. A method of treating a patient having rheumatoid arthritis, comprising:
administering sequentially to said patient, a therapeutically effective dose of a BTK inhibitor for a first period, followed by a therapeutically effect dose of abatacept for a second period.

2. The method according to claim 1 wherein said BTK inhibitor is an irreversible inhibitor.

3. The method according to claim 1, wherein said BTK inhibitor is branebrutinib.

4. The method according to claim 3, wherein said therapeutically effective dose of branebrutinib is from 1 to 10 mg per day.

5. The method according to claim 1, wherein abatacept is administered as a subcutaneous injection.

6. The method according to claim 1, wherein said therapeutically effective dose of abatacept is 125 mg per week.

7. The method according to claim 1, wherein said therapeutically effective dose of branebrutinib is from 1 to 10 mg per day; and said therapeutically effective dose of abatacept is 125 mg per week.
